(19) United States
(12) Patent Application Publication
(10) Pub. No.: US 2025/0186972 A1
(43) Pub. Date: Jun. 12, 2025

(54) MOLECULARLY IMPRINTED POLYMER

(71) Applicant: UNIVERSITEIT MAASTRICHT, MAASTRICHT (NL)

(72) Inventor: Joseph William LOWDON, MAASTRICHT (NL)

(21) Appl. No.: 18/846,156

(22) PCT Filed: Apr. 5, 2023
(86) PCT No.: PCT/EP2023/059014

§ 371 Date: Sep. 11, 2024

(30) Foreign Application Priority Data
Apr. 11, 2022 (EP) 22167571.3

(51) Int. Cl. B01J 20/26; B01J 20/28; G01N 33/546

(52) U.S. Cl. CPC B01J 20/268; B01J 20/267; B01J 20/28016; G01N 33/546

(57) ABSTRACT

The invention relates to a molecularly imprinted polymer, wherein the molecularly imprinted polymer comprises a functionalized polymer composition configured to molecularly bind a target molecule in a medium, wherein a chelating agent is molecularly bound to the functionalized polymer composition of the molecularly imprinted polymer, said chelating agent is releasable to the medium and is able to induce a change in the electrochemical properties of the medium once released from the molecularly imprinted polymer, and wherein the chelating agent is configured to be released to the medium from the molecularly imprinted polymer by molecular binding of the target molecule to the functionalized polymer composition of the molecularly imprinted polymer.

### TECHNICAL FIELD

The invention relates to a molecularly imprinted polymer, method of performing a molecularly imprinted polymer displacement assay and a sensor device comprising the molecularly imprinted polymer of the present invention for performing the method of the present invention. Further, the present invention relates to the use of the molecularly imprinted polymer in the detection and/or quantification of a target molecule. The invention further relates to a method of preparing the molecularly imprinted polymer of the present invention.

### BACKGROUND

Molecularly imprinted polymers (MIPs) are polymeric materials containing microscale cavities or imprints of defined shape. To create the imprints, target molecules are introduced into a solution containing polymerizable molecules that bind to the target molecules. Next, reaction conditions are changed, or crosslinking reagents are added to the solution, to cause the polymerizable molecules to form a solid polymer matrix in which the target molecules are immobilized. Finally, the target molecules are removed from the polymer matrix to form imprints having a particular shape. The MIP thus formed is able to selectively bind molecules that match the imprint shape with a lock-and-key-type interaction, when exposed to an environment containing a mixture of compounds.

Typically, molecularly imprinted polymer-based sensors comprise a molecularly imprinted polymer particles deposited onto a flat substrate surface forming a receptor layer for binding target molecules of a sample. The deposition of the MIP layer onto the flat substrate surface entails fixing of the MIP particles to the substrate using an adhesive or by chemically binding the MIP particles to the flat substrate surface. The process of depositing MIP particles onto the flat substrate surface is heterogenous and intrasample variance is high as MIP particle size variation will lead to different sensitivity of the sensor at specific spots of the flat substrate surface. In addition, the flat substrate surface deposited thereupon MIP particles is placed inside the sensor next to or on a transducer. Measurements are typically conducted across the receptor layer, converting a binding event, i.e. the binding of a target molecule of the sample to the MIP particles, into a tangible property (e.g. change in mass, change in impedance, change in thermal resistance, and the like).

An example of an imprinted polymer-based sensor known in the art is depicted (schematically) in FIG. 1. FIG. 1 showing a conductive surface (the flat substrate surface) provided with a MIP receptor layer deposited onto the conductive surface. The receptor layer is placed inside the sensor comprising a gold electrode in order to measure a change in impedance. Given the imprinted polymer-based sensor known in the art (and depicted in FIG. 1) it is noted that the sensor design is complex as the receptor layer is placed next to or on the transducer. Consequently, the sensor design is limited by the dimensions of the conductive surface and position to the transducer, resulting in a limited surface area of the receptor layer able to interact with the target molecule comprised in a sample to be analysed.

In addition to the drawbacks provided above, it is further noted that the sensitivity of the imprinted polymer-based sensor known in the art is limited by the tangible property (e.g. change in mass, change in impedance, change in thermal resistance, and the like) measured due to the binding of the target molecule to the MIP receptor layer.

### SUMMARY OF THE INVENTION

In order to reduce the complexity, to improve the design flexibility and/or to increase the sensitivity of imprinted polymer-based sensors known to date the present invention provides hereto a molecularly imprinted polymer, wherein the molecularly imprinted polymer comprises a functionalized polymer composition configured to molecularly bind a target molecule in a medium, and wherein a chelating agent is molecularly bound to the functionalized polymer composition of the molecularly imprinted polymer. The chelating agent molecularly bound to the functionalized polymer composition of the molecularly imprinted polymer of the present invention is releasable to the medium and is, subsequently, able to induce a change in the electrochemical properties of the medium once released from the molecularly imprinted polymer. In order to correlate the change in the electrochemical properties of the medium and the detection and/or quantification of the target molecule, the chelating agent is configured to be released to the medium from the molecularly imprinted polymer by molecular binding of the target molecule to the functionalized polymer composition of the molecularly imprinted polymer.

It was found that by providing the molecularly imprinted polymer of the present invention, the present invention provides a methodology wherein the displacement of a chelating agent (in case of a binding event of the target molecule to the functionalized polymer composition of the molecularly imprinted polymer) results in a change of electrochemical property of the medium wherein the chelating agent is released in. For example, in case an ion-binding chelating agent is used in the molecularly imprinted polymer of the present invention, the chelating agent will interact with the ions comprised in the medium. The ion-chelating agent interaction consequently results in a change of electrical potential of the medium. Instead of measuring the actual binding of a target molecule to the molecularly imprinted polymer of the present invention, an indirect measurement is herewith provided, leading to a higher sensitivity of a sensor based on the molecularly imprinted polymer of the present invention.

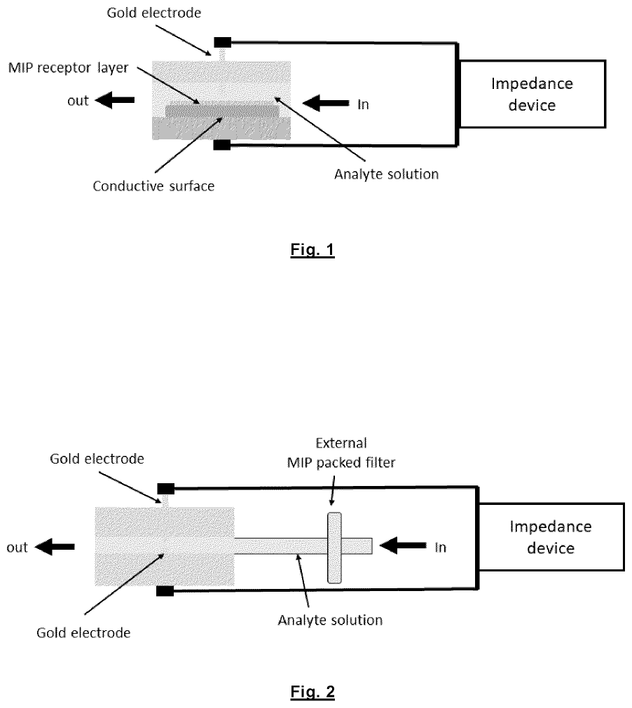

By measuring the changing electrochemical properties of the medium comprising the target molecule, there is no further need of measuring an actual binding event of the target molecule with a receptor layer comprised in a sensor as such. In other words, the measuring of the changing electrochemical properties of the medium may be performed (and is preferably performed) remote from the actual target molecule-MIP interaction site. Resulting in a less complex sensor design. An example of such simplified sensor design comprising the molecularly imprinted polymer of the present invention is depicted (schematically) in FIG. 2. FIG. 2 shows a measuring device (sensor comprising gold electrodes) in relation to which a filter packed with the molecularly imprinted polymer of the present invention is arranged upstream. A sample medium is allowed to flow through the MIP-packed filter to the measuring device. By binding of a target molecule comprised in the sample medium, the release of chelating agent is facilitated and results in a property change of the medium released from the filter. The property change of the medium is subsequently measured by the measuring device located at some distance from the filter packed with the molecularly imprinted polymer of the present invention.

Given the molecularly imprinted polymer of the present invention, as there is no further need of depositing the molecularly imprinted polymer of the present invention on a substrate surface, any problems in relation to the heterogenous sensor design and intrasample variance are solved as well. The present invention provides for a sensor based on the molecularly imprinted polymer of the present invention, wherein the molecularly imprinted polymer of the present invention maybe packed into a column and/or placed onto a filter that is remote from the actual sensor (i.e. medium properties measuring device). As such in designing a sensor based on the molecularly imprinted polymer of the present invention, the design of the sensor is no longer limited by the surface area of the substrate inside the sensor device as known to date.

It is further noted that by providing the molecularly imprinted polymer of the present invention, the present invention now provides for a MIP based sensor wherein the binding site of the target molecule to the MIP receptor layer is no longer two dimensional but three dimensional instead. Such three dimensional design results in a significant increase in interacting surface area of the molecularly imprinted polymer of the present invention with the target molecule of a sample, thus facilitating a significant increase in the sensitivity of the sensor based on the molecularly imprinted polymer of the present invention.

Also, given the molecularly imprinted polymer of the present invention, it is noted that the present invention now provides for an inline sensing methodology and/or continues sensing methodology. Even further, as the deposition step of depositing the molecularly imprinted polymer layer onto the substrate surface is no longer necessary/applicable, the molecularly imprinted polymer of the present invention is commercially more attractive including less expensive production costs.

### DETAILED DESCRIPTION OF THE INVENTION

As provided above, in a first aspect the present invention relates to a molecularly imprinted polymer, wherein the molecularly imprinted polymer comprises a functionalized polymer composition configured to molecularly bind a target molecule in a medium, and wherein a chelating agent is molecularly bound to the functionalized polymer composition of the molecularly imprinted polymer. The chelating agent molecularly bound to the functionalized polymer composition of the molecularly imprinted polymer of the present invention is releasable to the medium and is, subsequently, able to induce a change in the electrochemical properties of the medium once released from the molecularly imprinted polymer. In order to correlate the change in the electrochemical properties of the medium and the detection and/or quantification of the target molecule, the chelating agent is configured to be released to the medium from the molecularly imprinted polymer by molecular binding of the target molecule to the functionalized polymer composition of the molecularly imprinted polymer. Preferably, the molecularly imprinted polymer of the present invention may be suited for use in a displacement assay, wherein the chelating agent of the molecularly imprinted polymer is configured to be released to the medium from the molecularly imprinted polymer by displacement of the chelating agent with the target molecule.

As used herein, the term “functionalized polymer composition” refers to synthetic polymers that have been tailored to selectively bind a particular compound or a combination of particular compounds. The functionalized polymer composition is synthesized in the presence of target compounds, also referred to as template compounds, creating a MIP with a high degree of affinity for the specific target compound. Generally, the polymers are constructed with ligands spatially orientated forming the cavities that conform to the shapes of the associated target compounds. Specifically, the target compounds are incorporated into a pre-polymeric mixture and allowed to form bonds with the ligands. The mixture is then polymerized with the target compounds in place. Once the polymer has formed, the target compounds are removed, leaving behind cavities corresponding to the target compounds. Such cavities are thus tailored for binding future target compounds.

It is worthy to note, that while specific target compounds are used to form functionalized polymer compositions, the polymers may have a high affinity for a class of compounds that is similar to the target compound. A molecularly imprinted polymer is thus provided that may bind a number of compounds that are similar in shape, charge density, geometry or other physical or chemical properties.

In particular, the functionalized polymer composition of the present invention preferably comprises monomers comprising one or more functional groups crosslinked with crosslinkers comprising one or more functional groups.

As used herein, the terms “molecular bind”, “molecular binding” or “binding event” refers to the interaction of the functionalized polymer composition with the chelating agent or target molecule. Such binding events may also referred to as “selective binding characteristics” and “selective binding interactions” and are intended to refer to preferential and reversible binding exhibited by an imprinted polymer for its imprint molecule compared to other non-imprint molecules. Selective binding includes both affinity and specificity of the imprinted polymer for its template molecule and towards an analyte to be detected by a sensor device based on the molecularly imprinted polymer of the present invention (i.e. a “target molecule” or “target analyte”).

As used herein, the term “chelating agent” or “chelant” refers to sequestering agents and the like able to form chelate complexes with the chelating agent in order to induce a change of the electrochemical properties of the medium comprising the target molecule, e.g. by forming chelate complexes with the chelating agent and ions present in the medium. The chelating agent of the present invention is preferably able to interact with ions in the medium comprising the target molecule, i.e. to form a chelate with ions comprised in the medium. Preferably, wherein the ions comprised in the medium are metal ions.

Preferably, the term “chelating agent” as used herein, refers to a molecule containing two or more electron donor atoms that can form coordinate bonds to a single metal ion. The term “chelating agent” is understood to include the chelating agent as well as salts thereof. For example, the term “chelating agent” includes citric acid as well as its salt forms.

The most common and widely used chelating agents coordinate to metal atoms through oxygen or nitrogen donor atoms, or both. Other less common chelating agents coordinate through sulphur in the form of —SH (thiol or mercapto) groups. After the first coordinate bond is formed, each successive donor atom that binds creates a ring containing the metal atom. A chelating agent may be bidentate, tridentate, tetradentate, etc., depending upon whether it contains two, three, four, or more donor atoms capable of binding to the metal atom including Mg²⁺, Cu²⁺, Cu⁺, Ca²⁺, Hg²⁺, Hg⁺, Fe³⁺, Fe²⁺, or the like. By binding of the metal atom comprised in the medium comprising the target molecule, the chelating binding interaction can be monitored with a readout technology including impedance (conductivity/resistivity), electrical potential, colour change (by use of an indicator dye) or the like, i.e. monitoring the electrochemical properties, e.g. the ionic properties, of the medium.

Suitable chelating agents that may be used in the molecularly imprinted polymer of the present invention are preferably non-colouring chelating agents and/or non-toxic chelating agents. In particular, preferably the chelating agent molecularly bound to the functionalized polymer composition of the molecularly imprinted polymer of the present invention does not comprise a dye molecule.

Suitable chelating agents may be selected from the group consisting of (but are not limited to) ethylenediaminetetraacetic acid (EDTA) based chelating agents, dimercaprol based chelating agents, citric acid, 2,2′-bis(diphenylphosphino)-1,1′-binaphthyl) (BINAP) based chelating agents, and phosphonate based chelating agents.

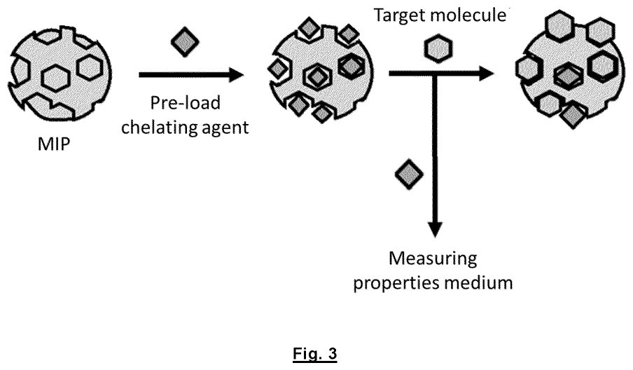

According to the present invention, a molecularly imprinted polymer is provided wherein the chelating agent is molecularly bound to the functionalized polymer composition of the molecularly imprinted polymer. In order to provide the molecularly imprinted polymer of the present invention, the molecularly imprinted polymer is pre-loaded with a suitable chelating agent. As used herein, the term “pre-loaded” is intended to refer to the method of incubating over a sufficient period of time the functionalized polymer composition with the chelating agent, followed by washing the incubated molecularly imprinted polymer until no chelating agent can be detected in the washing medium. The pre-loading thus results in a molecularly imprinted polymer wherein all or at least the vast majority (i.e. more than 90%, preferably more than 95%) of the cavities of the functionalized polymer composition are provided (or occupied) with the chelating agent. In this respect reference is made to FIG. 3 (schematically) showing the incubation or pre-loading of the functionalized polymer composition with the chelating agent.

As used herein, the term “medium” may refer to a liquid, biological fluid or sample which may contain the target molecule. Preferably, the medium according to the present invention is an aqueous medium.

The molecularly imprinted polymer of the present invention may be in the form of particles, such as powders, granules, beads, crystals, pellets and the like. By providing such a molecularly imprinted polymer, the present invention facilitates the packing of the molecularly imprinted polymer into columns, resulting in a significant increase (and consequently significant increase in sensitivity) in interacting surface area of the molecularly imprinted polymer of the present invention with the target molecule of a sample.

In a second aspect the present invention relates to a method of performing a molecularly imprinted polymer displacement assay, wherein the method comprises the steps of:
a) providing the molecularly imprinted polymer according to the present invention;
b) providing a medium comprising the target molecule;
c) incubating the molecularly imprinted polymer provided in step a) with the medium provided in step b); and
d) monitoring electrochemical properties of the medium.

As used herein, the term “electrochemical properties of the medium” may refer to the impedance, electric potential, conductivity and resistivity of the medium. Also, the term “electrochemical properties of the medium” may refer to colour change of an ion indicator dye comprised in the medium. In this respect, it is noted that the monitoring of the electrochemical properties of the medium in step d) may include monitoring the impedance, electric potential, conductivity, resistivity, colour change of an ion indicator dye comprised in the medium and combinations thereof.

In case an ion indicator dye is used in the displacement assay of the present invention, the ion indicator dye has preferably a lower ion affinity compared to the ion affinity of the chelating agent molecularly bound (i.e. pre-loaded) to the molecularly imprinted polymer.

Step c) of incubating the molecularly imprinted polymer with the medium may comprise the step of:
allowing a predetermined amount of medium to act upon the molecularly imprinted polymer; or
allowing a flow of medium to flow through or over the molecularly imprinted polymer.

The method of performing the displacement assay provided by the present invention is suitable for the application of a single sample onto the molecularly imprinted polymer particles of the present invention. Also the method of performing the displacement assay provided by the present invention is suitable for the continuous or intermittent monitoring of a flow of medium flowing through or over the molecularly imprinted polymer of the present invention.

In addition, the method of performing the displacement assay of the present invention may further comprise the step of:
e) quantifying the target molecule based on a change in electrochemical properties monitored in step d).

In a third aspect the present invention relates to a sensor device for sensing a target molecule in a medium, wherein the sensor device comprises the molecularly imprinted polymer according to the first aspect of the present invention for performing the method according to the second aspect of the present invention.

The sensor device may comprise an inlet for feeding the medium to the sensor device and an outlet for discharging the medium from the sensor device. The sensor device may further comprises a monitoring unit for monitoring the electrochemical properties of the medium.

In a preferred embodiment of the present invention, the monitoring unit of the sensor device is arranged downstream the molecularly imprinted polymer. By providing such arrangement, the design of the sensor device is less complex. Also, the flexibility and capacity (e.g. sensitivity) of the sensor device is further improved by providing a monitoring unit arranged downstream the molecularly imprinted polymer of the present invention.

In a fourth aspect the present invention relates to the use of the molecularly imprinted polymer according to the present invention in the detection and/or quantification of a target molecule in a medium. For example, the present invention relates to the use of the molecularly imprinted polymer according to the present invention in a molecularly imprinted polymer displacement assay as described above.

In a fifth aspect the present invention relates to a method of preparing the molecularly imprinted polymer according to the present invention, wherein the method comprises the steps of:
i) preparing the functionalized polymer composition;
ii) incubating the functionalized polymer composition with the chelating agent; and
iii) drying the functionalized polymer composition incubated with chelating agent to form the molecularly imprinted polymer.

The method of the present invention may further comprise the step of:
after incubating the functionalized polymer composition with the chelating agent in step ii) and before drying the functionalized polymer composition incubated with chelating agent to form the molecularly imprinted polymer in step iii), washing the incubated functionalized polymer composition to remove the excess of chelating agent from the incubated functionalized polymer composition.

### Examples

### Synthesis and Analysis of Molecularly Imprinted Polymers

The synthesis and analysis of the amoxicillin molecularly imprinted polymer was carried out as described in previous research by Lowdon et al (“Colorimetric Sensing of Amoxicillin Facilitated by Molecularly Imprinted Polymers.” Polymers 13.13 (2021): 2221).

### Synthesis of Molecularly Imprinted Polymers

An aqueous phase was prepared by mixing 300 mg of sodium dodecyl sulphate (SDS) in 60 mL of DI water and placing it into a 100 mL R.B.F. An overhead stirrer was then placed into the flask, with the stirrer blade positioned so that it was just about covered by the solution. Proceeding this the organic phase of the reaction was prepared by mixing monomer (methacrylic acid), EGDMA, AIBN, amoxicillin and DMSO in a vial before purging both solutions with N₂ for 15 minutes. Once purged, the organic phase was introduced to the aqueous phase while being vigorously stirred between 600-1400 rpm for 1 minute enabling an emulsion to form. Stirring was ceased, and the emulsion exposed to UV light (BlueWave 200) for 1 hour. After the exposure the solution was transferred to centrifuge tubes and spun at 4500 rpm for 5 minutes. The resulting polymeric disk was left in the centrifuge tube while carefully removing the supernatant, and introducing ethanol to the tube. The tube was well shaken to suspend the polymeric powder and the centrifugation process was then repeated a further 5 times. Once fully washed the polymeric powder was transferred into a glass vial and oven dried at 65° C. for 12 hours. Thus yielding a fine polymeric powder with a defined particle size based on the rpm initially used to mix the reaction. A non-imprinted reference composition was synthesized with the exact sample methodology but without the presence of the template molecule. Template extraction was monitored with FTIR comparing the extracted polymeric composition spectra to that of the amoxicillin.

### Binding Experiments (Amoxicillin)

To determine the binding affinities of the MIP and non-imprinted polymers (NIP), rebinding experiments were conducted as follows: to 20 mg of MIP/NIP powder was added 5 mL aliquots of aqueous molecular species (amoxicillin) ranging in concentration (0.1-0.7 mM) with the resulting suspension agitated on a rocking table (125 rpm) for 90 minutes. After agitation, the filtrate of each sample was collected and analyzed using a Shimadzu 3600 UV-spectrophotometer, analyzing the λₘₐₓ of the remaining molecular species in solution (C_{f}). The amount of molecular species bound to the MIP/NIP (S_{b}) proceeded to be calculated from these observed values and the corresponding binding isotherm plotted.

### Binding Experiments (EDTA)

The experiments regarding the binding of EDTA was carried out much in the same manner as with amoxicillin, though with some slight modifications: to 20 mg of MIP/NIP powder was added 5 mL aliquots of aqueous molecular species (EDTA) ranging in concentration (0.1-0.7 mM) with the resulting suspension agitated on a rocking table (125 rpm) for 90 minutes. After agitation, the filtrate of each sample was collected and analysed by means of complexometric titration. In essence, the filtrate was titrated against aqueous calcium chloride (0.01 mM) in the presence of an Eriochrome black T indicator dye (Preparation of indicator dye below) with the end point of the titration being determined by the colour of the solution shifting from blue to pink. The concentration of the EDTA present in solution was therefore determined by the volume of the titrated calcium chloride solution. This metric was then used to calculate the amount of EDTA bound to the MIP/NIP and construct the relevant binding isotherm was done in the previous binding experiments.

### Preparation of Eriochrome Black T Indicator Solution

0.5 g of Eriochrome black T was dissolved in 50 mL of ethyl alcohol and was buffered with a few drops of ammonia-ammonium chloride buffer (preparation: Dissolve 67.5 g of ammonium chloride in 200 mL of water, add 570 mL of ammonia solution and dilute with water to 1000 mL).

### Preparation of Aluminium Substrate

Samples were prepared in accordance with Caldara et al (“Thermal Detection of Glucose in Urine Using a Molecularly Imprinted Polymer as a Recognition Element.” ACS sensors 6.12 (2021): 4515-4525). Aluminium plates were polished and cut to obtain the desired dimensions (1×1×0.5 cm²). To immobilize MIP particles, a PVC adhesive layer (4 wt % PVC dissolved in tetrahydrofuran) was deposited on the aluminium chip by spin coating (2000 rpm for 60 s with an acceleration of 1000 rpm s⁻¹). To stamp the particles on the PVC layer, a PDMS substrate, covered with a monolayer of MIP particles, was used. The PVC layer was heated for 2 h at a temperature above its glass transition temperature (100° C.), allowing the beads to sink into the polymer layer. The samples were cooled down prior to thermal measurements, and any unbound particles were washed off with distilled water.

### Impedimetric Analysis of MIPs

For the analysis of the traditional receptor layer (inside the flow cell), impedimetric analysis was performed as described by Arreguin-Campos et al (“Biomimetic sensing of Escherichia coli at the solid-liquid interface: From surface-imprinted polymer synthesis toward real sample sensing in food safety.” Microchemical Journal 169 (2021): 106554) using a MFIA impedance analyzer from Zurich Instruments and replica flow cell. Continuous frequency sweeps of 200 points were taken across a 10-500,000 Hz range, with a test signal of 300 mV. The initial signal was stabilized in a solution containing calcium chloride (1.6 M) for 10 minutes before the introduction of an amoxicillin solution (2 mL) into the flow cell at a rate of 4 mL min⁻¹, before allowing stabilization for a further 10 minutes. This introduction was repeated across a range of amoxicillin concentrations (10 nM-1 mM), ensuring that all solutions were prepared in the same concentration electrolyte and the same stabilization period was followed.

### Preparation of EDTA Bound MIPs

The binding of EDTA to the amoxicillin MIP was conducted by incubated 0.5 g of MIP powder with 50 mL of EDTA solution (1 mM) for 2 hours. After this period had elapsed the EDTA bound polymer particles were then filter gravimetrically, before rinsing the collected powder with 5×50 mL of DI water. After each wash the filtrate was collected and the EDTA concentration present analyzed by means of colorimetric titration (see the supplementary section for methodology and analysis results), ensuring that any unbound or weakly bound EDTA was removed from the polymer before drying at 65° C. overnight.

### Preparation of Receptor for Impedimetric Analysis

As the EDTA bound MIPs did not require deposition, they were instead loaded onto a PTFE filter by means of suspending 25 mg of EDTA bound MIP particles in DI water and passing them through a PTFE (0.45 μm pore size) filter for ease of use.

### Impedimetric Analysis (EDTA Displacement Technique)

The same procedure as described above was conducted for the EDTA displacement that occurs when amoxicillin is incubated with the EDTA bound MIPs, though some experimental differences were made. Firstly, as there was no need for the receptor inside the flow cell, the substrate inside was replaced by another gold electrode (0.5 mm diameter) that was placed parallel to the gold electrode already installed. Secondly, a 3D printed polyacrylate-based lid, ensuring the dimensions and volume remained consistent with the first, replaced the copper block. Finally, as the receptor no longer sat inside the flow cell, a filter with EDTA bound MIPs was connected to the inlet tubing instead. The rest of the produced remained the same, with the same concentration range of amoxicillin (0.01 nM-100 nM) investigated for direct comparison.

### Results

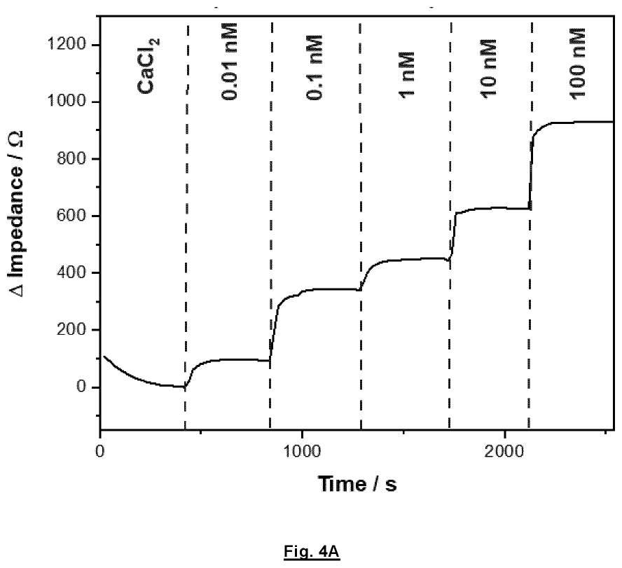

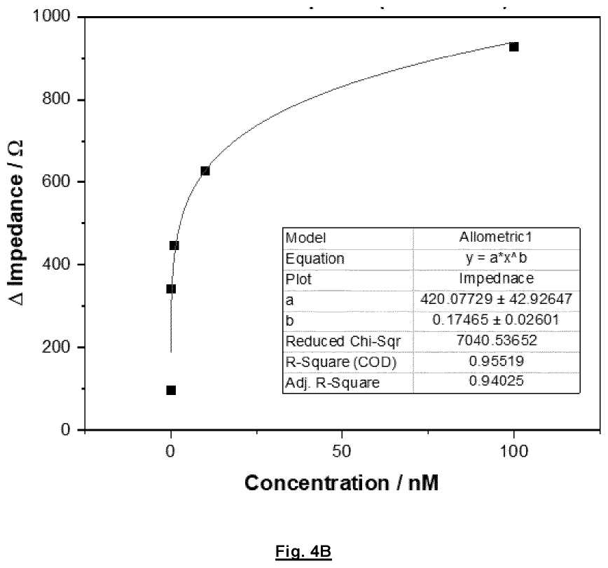

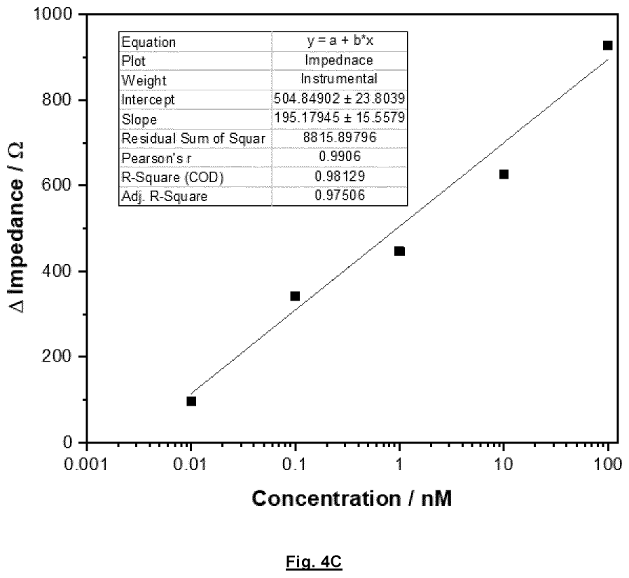

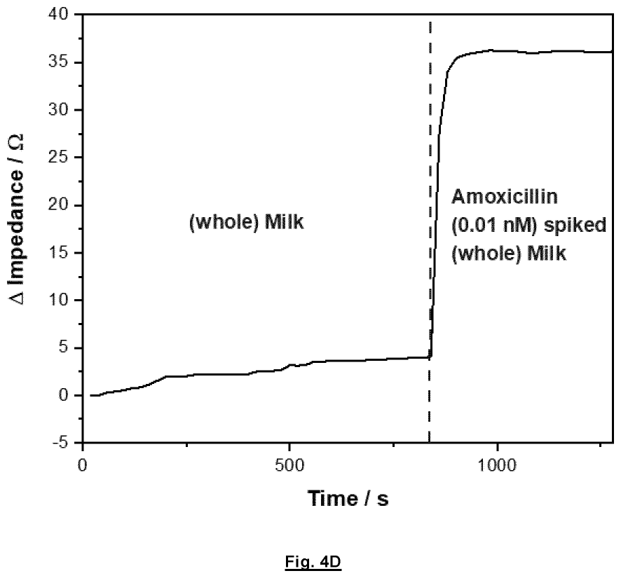

Impedance analysis of the assay was conducted while exposing the sensor to increasing concentrations of amoxicillin (the target molecule) in solution. FIG. 4A shows the raw change in impedance versus time observed when a concentration range of 0.01 nM to 100 nM of amoxicillin in solution was added across the sensor. Initially the system was stabilized in a calcium chloride solution (1.6 mM to simulate the concentration in cow's milk). As shown in FIG. 4A with each concentration infusion there is an observed change in impedance, indicating that the chelating agent is interacting with ions in the solution, and therefore leading to a change in the impedance.

Subsequently, the concentration was then plotted against the change in impedance (FIG. 4B), demonstrating the relationship between the two variables (linear x-axis scale), whereas in FIG. 4C the relationship between concentration and change in impedance is shown when a logarithmic x-axis scale is introduced.

FIG. 4D shows the results of an initial experiment to see if the sensor works in a complex environment. In this experiment, the sensor was stabilized in (whole) milk before introducing a spiked milk sample (comprising 0.01 nM amoxicillin). As depicted in FIG. 4D a clear change in impedance is observed. As indicated by the clear change of impedance in FIG. 4D, more chemically complex samples can be analysed with the MIPs of the present invention.

Given the above, it is further noted that the experiments were conducted by passing liquid samples through a filter where 25 mg of EDTA loaded MIP powder was situated. It is however noted that the amount of MIP powder present on the filter can be increased, increasing the linear range of the sensor and also further improving the sensitivity of the method. As a consequence, the sensor of the present invention can be applicable towards greater concentrations ranges (1 picomolar to 1 millimolar).

1. Molecularly imprinted polymer, wherein the molecularly imprinted polymer comprises a functionalized polymer composition configured to molecularly bind a target molecule in a medium,
characterized in that a chelating agent is molecularly bound to the functionalized polymer composition of the molecularly imprinted polymer, said chelating agent is releasable to the medium and is able to induce a change in the electrochemical properties of the medium once released from the molecularly imprinted polymer, and
in that the chelating agent is configured to be released to the medium from the molecularly imprinted polymer by molecular binding of the target molecule to the functionalized polymer composition of the molecularly imprinted polymer.

2. Molecularly imprinted polymer according to claim 1, wherein the chelating agent is configured to be released to the medium from the molecularly imprinted polymer by displacement of the chelating agent with the target molecule.

3. Molecularly imprinted polymer according to claim 1, wherein the chelating agent is able to form a chelate with ions comprised in the medium, preferably wherein the ions comprised in the medium are metal ions.

4. Molecularly imprinted polymer according to claim 1, wherein the chelating agent does not comprise a dye molecule, preferably wherein the chelating agent is selected from the group consisting of ethylenediaminetetraacetic acid based chelating agents, dimercaprol based chelating agents, citric acid, 2,2′-bis(diphenylphosphino)-1,1′-binaphthyl based chelating agents and phosphonate based chelating agents.

5. Molecularly imprinted polymer according to claim 1, wherein the polymer composition is in the form of particles, such as powders, granules, beads, crystals, pellets and the like.

6. Molecularly imprinted polymer according to claim 1, wherein the functionalized polymer composition comprises monomers comprising one or more functional groups crosslinked with crosslinkers comprising one or more functional groups.

7. Method of performing a molecularly imprinted polymer displacement assay, wherein the method comprises the steps of:
a) providing the molecularly imprinted polymer according to claim 1;
b) providing a medium comprising the target molecule;
c) incubating the molecularly imprinted polymer provided in step a) with the medium provided in step b); and
d) monitoring electrochemical properties of the medium.

8. Method according to claim 7, wherein the electrochemical properties of the medium monitored in step d) include impedance, electric potential, conductivity, resistivity, colour change of an ion indicator dye comprised in the medium and combinations thereof.

9. Method according to claim 8, wherein the ion indicator dye has a lower ion affinity compared to the ion affinity of the chelating agent molecularly bound to the molecularly imprinted polymer.

10. Method according to claim 7, wherein step c) of incubating the molecularly imprinted polymer with the medium comprises the step of:
allowing a predetermined amount of medium to act upon the molecularly imprinted polymer; or
allowing a flow of medium to flow through or over the molecularly imprinted polymer.

11. Method according to claim 7, wherein the assay further comprises the step of:
e) quantifying the target molecule based on a change in electrochemical properties monitored in step d).

12. Sensor device for sensing a target molecule in a medium, wherein the sensor device comprises the molecularly imprinted polymer according to claim 1.

13. Sensor device according to claim 12, wherein the sensor device comprises an inlet for feeding the medium to the sensor device and an outlet for discharging the medium from the sensor device, and wherein the sensor device further comprises a monitoring unit for monitoring the electrochemical properties of the medium, said monitoring unit is arranged downstream the molecularly imprinted polymer.

14. Use of the molecularly imprinted polymer according to claim 1 in the detection and/or quantification of a target molecule in a medium.

15. Method of preparing the molecularly imprinted polymer according to claim 1, wherein the method comprises the steps of:
i) preparing the functionalized polymer composition;
ii) incubating the functionalized polymer composition with the chelating agent; and
iii) drying the functionalized polymer composition incubated with chelating agent to form the molecularly imprinted polymer.
